SEQUENCE LISTING (12 sequences)

SEQ ID NO: 1 - DNA, 10372 bp, Nucleotide sequence of V3 embodiment
tggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcctgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtaagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaggctatacatgggcaggcaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttctggaagaactgaaagaagaagctttaaaacattttgatcctcgcttgctaactgcacttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaatggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaagatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcattgttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccaacaacaatacaagaaaaagaatccgtatccagagaggaccagggagagcatttgttacaatgggaaaaataggagatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtattggcaacaatgagttcgagatcttcagacctggaggaggagatatgaaggacaattggagaagtgaattatataaatataaagtagtaaaaattgaaccattaggagtagcacccaccaaggcaaagagaagagtggtgcagagagaaaaaagagcagtgggaataggagctgtgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaatcagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccacagccatagcagtagctgagaggacagatagggttatagaagtagtacaaggagcttgtagagctattcgccacatacctagaagaataagacagggcttggaaaggattttgctataagacggaccatggagaaacccagctgaagagaaagaaaaattagcatacagaaaacaaaatatggatgatatagatgaggaagatgatgacttggtaggggtatcagtgaggccaaaagttcccctaagaacaatgagttacaaattggcaatagacatgtctcattttataaaagaaaaggggggactgggagggatttattacagtccaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcccagaaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaagctagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 2 - DNA, 9232 bp, Nucleotide sequence of V4 embodiment
tggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcctgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtaagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagcacacaaaggaggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaggctatacatgggcaggcaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttctggaagaactgaaagaagaagctttaaaacattttgatcctcgcttgctaactgcacttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaatggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaagatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcattgttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccaacaacaatacaagaaaaagaatccgtatccagagaggaccagggagagcatttgttacaatgggaaaaataggagatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtattggcaacaatgagttcgagatcttcagacctggaggaggagatatgaaggacaattggagaagtgaattatataaatataaagtagtaaaaattgaaccattaggagtagcacccaccaaggcaaagagaagagtggtgcagagagaaaaaagagcagtgggaataggagctgtgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaatcagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccacagccatagcagtagctgagaggacagatagggttatagaagtagtacaaggagcttgtagagctattcgccacatacctagaagaataagacagggcttggaaaggattttgctataagacggaccatggagaaacccagctgaagagaaagaaaaattagcatacagaaaacaaaatatggatgatatagatgaggaagatgatgacttggtaggggtatcagtgaggccaaaagttcccctaagaacaatgagttacaaattggcaatagacatgtctcattttataaaagaaaaggggggactgggagggatttattacagtccaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcccagaaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaagctagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 3 - DNA, 9876 bp, Nucleotide sequence of V5 embodiment
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagacaatatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 4 - DNA, 2039 bp, Nucleotide sequence of vector pET-9a
gaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattc

SEQ ID NO: 5 - DNA, 9984 bp, Nucleotide sequence of V6 embodiment
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaat

SEQ ID NO: 6 - DNA, 9715 bp, Nucleotide sequence of V7 embodiment
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacgcgaaagcgaaagtagaaccagaggagctctctcgacgcaggactcggcttgctgaagcgcgcacagcaagaggcgaggggcggcgactggtgagtacgccaatttttgactagcggaggctagaaggagagagagatgggtgcgagagcgtcggtattaagcgggggagaattagataaatgggaaaaaattcggttaaggccagggggaaagaaaaaatataagttaaaacatatagtatgggcaagcagggagctagaacgattcgcagtcaatcctggcctgttagaaacatcagaaggctgcagacaaatattgggacagctacagccatcccttcagacaggatcagaagaacttagatcattatataatacagtagcaaccctctattgtgtacatcaaaggatagatgtaaaagacaccaaggaagctttagagaagatagaggaagagcaaaacaaaagtaagaaaaaggcacagcaagcagcagctgcagctggcacaggaaacagcagccaggtcagccaaaattaccctatagtgcagaacctacaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagactatcaatgaggaagctgcagaatgggatagagtgcatccagtgcatgcagggcctattgcaccaggccaaatgagagaaccaaggggaagtgacatagcaggaactactagtacccttcaggaacaaataggatggatgacaaataatccacctatcccagtaggagaaatctataaaagatggataatcctgggattaaataaaatagtaagaatgtatagccctaccagcattctggacataagacaaggaccaaaggaaccctttagagattatgtagaccggttctataaaactctaagagccgaacaagcttcacaggatgtaaaaaattggatgacagaaaccttgttggtccaaaatgcaaacccagattgtaagactattttaaaagcattgggaccagcagctacactagaagaaatgatgacagcatgtcagggagtggggggacccggccataaagcaagagttttggctgaagccatgagccaagtaacaaatccagctaacataatgatgcagagaggcaattttaggaaccaaagaaagactgttaagtgtttcaattgtggcaaagaagggcacatagccaaaaattgcagggcccctaggaaaaagggctgttggagatgtggaagggaaggacaccaaatgaaagattgcactgagagacaggctaattttttagggaagatctggccttcctacaagggaaggccagggaattttcttcagagcagaccagagccaacagccccaccagaagagagcttcaggtttggggaggagaaaacaactccctctcagaagcaggagccgatagacaaggaactgtatcctttaacttccctcagatcactctttggcaacgacccctcgtcacaataaggataggggggcaactaaaggaagctctattagatacaggagcagatgatacagtattagaagaaatgaatttgccaggaaaatggaaaccaaaaatgatagggggaattggaggttttatcaaagtaagacagtacgatcagatacctgtagaaatctgtggacataaagctataggtacagtattagtaggacctacacctgtcaacataattggaagaaatctgttgactcagattggttgtactttaaatttccccattagtcctattgaaactgtaccagtaaaattaaagccaggaatggatggcccaaaagttaagcaatggccattgacagaagaaaaaataaaagcattagtagagatatgtacagaaatggaaaaggaagggaaaatttcaaaaattgggcctgaaaatccatacaatactccagtatttgctataaagaaaaaagacagtactaaatggagaaaactagtagatttcagagaacttaataaaagaactcaagacttctgggaagttcagttaggaataccacaccccgcagggttaaaaaagaaaaaatcagtaacagtattggatgtgggtgatgcatacttttcagttcccttagataaagactttagaaagtatactgcatttaccatacctagtataaacaatgagacaccagggattagatatcagtacaatgtgctgccacagggatggaaagggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgacatgggtggcaagtggtcaaaacgtagtatgggtggatggtctgctataagggaaagaatgagacgagctgagccacgagctgagccagcagcagatggggtgggagcagtatctcgagacctggaaaaacatggagcaatcacaagtagcaatacagcagctactaatgctgattgtgcctggctagaagcacaagaggaggaagaggtgggttttccagtcagacctcaggtacctttaagaccaatgacttacaaggcagctttagatattagccactttttaaaagaaaaggggggactggaagggctaatttggtcccaaagaagacaagagatccttgatctgtggatctaccacacacaaggctacttccctgattggcagaattacacaccagggccagggatcagatatccactgacctttggatggtgcttcaagctagtaccagttgagccagagaaggtagaagaggccaatgaaggagagaacaacagcttgttacaccctatgagcctgcatgggatggaggacgcggagaaagaagtgttagtgtggaggtttgacagcaaactagcatttcatcacatggcccgagagctgcatccggagtactacaaagactgctgagcggccgcctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaat

SEQ ID NO: 7 - DNA, 62 bp, Nucleotide sequence of deleted portion of vpu gene
atattaagacaaagaaaaatagacaggttaattgatagactaatagaaagagcagaagacag

SEQ ID NO: 8 - DNA, 216 bp, Nucleotide sequence of deleted portion of nef gene
cgacctacaatatgggtggagctatttccatgagacggtccaggccgtctggagatctgcgacagagactcttgcgggcgtgtggggagacttatgggagactcttaggagaggtggaagatggatactcgcaatccccaggaggattagacaagggcttgagctcactctcttgtgagggacagaaatacaatcaggaacagtatatgaatactc

SEQ ID NO: 9 - DNA, 762 bp, Nucleotide sequence of deleted portion of rt gene
agccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggatacc

SEQ ID NO: 10 - DNA, 86 bp, Nucleotide sequence of overlapping portion of vpu and env genes
atgagagtgaaggagaaatatcagcacttgtggagatgggggtggagatggggcaccatgctccttgggatgttgatgatctgtag

SEQ ID NO: 11 - DNA, 411 bp, Nucleotide sequence of portion of 3-prime LTR deleted in some embodiments
aacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 12 - DNA, 818 bp, Nucleotide sequence of portion of 3-prime LTR deleted in other embodiments
tggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

(19) United States
(12) Patent Application Publication
(10) Pub. No.: US 2003/0220276 A1
(43) Pub. Date: Nov. 27, 2003

(54) HIV vaccine and method of use

(76) Inventor: Opendra Narayan, Lenexa, KS (US)

Correspondence Address: BLACKWELL SANDERS PEPER MARTIN LLP, 720 OLIVE STREET, SUITE 2400, ST. LOUIS, MO, 63101, US

(21) Appl. No.: 10/279,992

(22) Filed: Oct. 24, 2002

Related U.S. Application Data
(63) Continuation-in-part of application No. 08/850,492, filed on May 2, 1997, now abandoned.
Application No. 08/850,492 is a continuation-in-part of application No. 08/442,010, filed on May 16, 1995, now Pat. No. 5,849,994.

(51) Int. Cl. A61K 48/00; A61K 39/21; C12N 15/867

(52) U.S. Cl. 514/44; 424/208.1; 435/456

(57) ABSTRACT

The present invention relates to a vaccine for immunization against HIV. The vaccine has DNA sequences encoding a plurality of viral proteins, including NEF, VPU and reverse transcriptase. The vaccine is rendered nonpathogenic by the disruption of the gene(s) encoding for at least one of these proteins.

### CROSS-REFERENCE TO RELATED APPLICATIONS

This application is a Continuation-in-part of U.S. patent application Ser. No. 08/850,492, filed May 2, 1997, which is, in turn, a Continuation-in-Part of U.S. patent application Ser. No. 08/442,010, filed May 16, 1995.

### STATEMENT OF GOVERNMENT RIGHTS

This work was supported in part by NIH grant numbers AI38492, RR06753 and RO1 AI51220; the Government of the United States of America may have rights in this invention.

### BACKGROUND OF INVENTION

The present invention relates generally to the field of prophylactic vaccines for generating protection from HIV-1 induced disease and infection. More specifically, the present invention relates to live virus and DNA vaccines against the Human Immunodeficiency Virus (HIV).

By the end of the year 2000, an estimated 36.1 million people worldwide were infected with HIV. In that year alone, HIV/AIDS-associated illnesses claimed the lives of approximately 3 million people worldwide. An estimated 500,000 of those deaths were of children under the age of fifteen. The importance of an HIV vaccine with respect to world health cannot be stated strongly enough.

It is recognized that effective vaccines which will inhibit or prevent HIV-1 infection or HIV-1-induced disease in humans will be useful for the treatment of certain high-risk populations, and as a general prophylactic vaccination for the general population that may risk HIV-1 infection or HIV-1-induced disease. A vaccine that will confer long-term protection against the transmission of HIV-1 would be most useful. Unfortunately, numerous problems stand in the way of developing effective vaccines for the prevention of HIV-1 infection and disease. Certain problems are most likely the result of the unique nature of the HIV-1 virus and its functional properties, and as yet no effective vaccine has been developed (for review see:

Berzofsky et al., Developing Synthetic Peptide Vaccines for HIV-1, Vaccines 95, pps. 135-142, 1995; Cease and Berzofsky, Toward a Vaccine for AIDS: The Emergence of Immunobiology-Based Vaccine Design, Annual Review of Immunology 12:923-989, 1994; Berzofsky, Progress Towards Artificial Vaccines for HIV, Vaccines 92, pps. 41-40, 1992).

HIV is a retrovirus, meaning that its genome consists of RNA rather than DNA. There are two primary strains of the virus, designated HIV-1 and HIV-2, with HIV-1 being the strain that is primarily responsible for human infection. The RNA genome of HIV is surrounded by a protein shell. The combination of the RNA genome and the protein shell is known as the nucleocapsid, which is in turn surrounded by an envelope of both protein and lipid.

Infection of host cells by HIV begins when the gp120 protein of HIV, a highly glycosylated protein located in the viral envelope, binds to the CD4 receptor molecule of the host cell. This interaction initiates a series of events that allow fusion between the viral and cell membranes and the subsequent entry of the virus into the cell.

Following entry into the host cell, HIV RNA is transcribed into double-stranded DNA by a viral reverse transcriptase enzyme. The HIV DNA is then integrated into the host cell genome by the action of the viral integrase enzyme. Once integrated into the host genome, HIV expresses itself through transcription by the host's RNA Polymerase II enzyme. Through both transcriptional control and postranscriptional transcript processing, HIV is able to exert a high level of control over the extent to which it expresses itself.

Studies of the HIV virus have revealed much information about the molecular biology of the virus, including information concerning a number of genes important to the pathogenicity of HIV. Four such genes are gag, pol, nef, and vpu.

The gag gene encodes for, among other things, the p27 capsid protein of HIV. This protein is important in the assembly of viral nucleocapsids. The p27 protein is also known to interact with the HIV cellular protein CyA, which is necessary for viral infectivity. Disruption of the interaction between p27 and CyA has been shown to inhibit viral replication.

The pol gene encodes viral enzymes important in enabling the virus to integrate into the host genome and replicate itself. The pol gene encodes, among other proteins, viral reverse transcriptase (RT) and integrase (IN). RT is an RNA-dependent DNA polymerase that synthesizes DNA from an RNA template. It is this enzyme that utilizes the RNA genome of HIV to produce a corresponding linear double-stranded DNA molecule that can be incorporated into the host genome. IN is the enzyme that actually catalyzes the insertion of the linear double-stranded viral DNA into the host cell chromosome.

The nef gene product (known as Negative Factor, or Nef) has a number of potentially important properties. Nef has the ability to downregulate CD4 and MHC Class I proteins, both of which are important to the body's ability to recognize virus-infected cells. Nef has also been shown to activate cellular protein kinases, thereby interfering with the signaling processes of the cell. Perhaps most importantly, deletion of nef from a pathogenic clone of Simian Immunodeficiency Virus (SIV) renders the virus nonpathogenic in adult macaque monkeys. Thus, a functional nef gene is crucial for the ability of SIV to cause disease in vivo. Further, studies have shown that HIV positive individuals with large deletions in the nef gene remained healthy for well over 10 years, with no reduction in cellular CD4 counts.

The vpu gene encodes a protein of originally unknown function (known as Viral Protein, Unknown, or Vpu), but which is now known to downregulate CD4 and MHC Class-I expression as well as promote viral budding. Vpu is also similar to another viral protein that acts as an ion channel. The vpu gene is present in HIV-1, but is absent in HIV-2.

Identical DNA sequences located at either end of the proviral DNA play an important role in the integration of HIV into the host genomes. These sequences are known as the 5′ and 3′ long-terminal repeats (LTR). In addition, transcription of HIV is mediated by a single promoter located within the 5′ LTR.

In nearly all viral infections, certain segments of the infected population recover and become immune to future viral infection by the same pathogen. Examples of typical viral pathogens include measles, poliomyelitis, chicken pox, hepatitis B, small pox, etc. The high mortality rate of HIV-1 infection, and the extremely rare incidence of recovery and protective immunity against HIV-1 infection, has cast doubt on the ability of primates to generate natural immunity to HIV-1 infection when pathogenic HIV-1 is the unmodified wild-type viral pathogen. Thus, there is a great need for a vaccine that will confer on primate populations protective immunity against HIV-1 virus.

A hallmark measure of resistance to future viral infection is the generation of ‘neutralizing antibodies’ capable of recognizing the viral pathogen. Another measure is cellular immunity against infected cells. In typical viral infections, generation of neutralizing antibodies and cellular immunity heralds recovery from infection. In HIV-1 infection, however, neutralizing antibodies and cellular immunity appear very early during the infection and have been associated with only a transient decrease in viral burden. In spite of the generation of neutralizing antibodies and cellular immunity, viral replication in HIV-1 infection rebounds and AIDS (acquired immune deficiency syndrome) develops. Thus, in HIV-1 infection neutralizing antibodies and cellular immunity are not accurate measures of protective immunity.

A further problem in developing an effective vaccine for HIV-1 is the antigenic diversity of the wild-type virus. There is a strong possibility that vaccines generated via recombinant HIV-1 coat proteins will confer resistance to specific phenotypes of virus and not broad spectrum immunity. Vaccine development using recombinant HIV-1 gp120 peptide, an HIV-1 viral coat protein, has passed phase-one clinical trials showing no toxicity. Data has indicated, however, that neutralizing antibodies appeared only transiently. Thus, recombinant HIV-1 gp12-peptide vaccines may act only in the short-term, with reversion to susceptibility of infection occurring in the future.

In general, it is accepted that live-virus vaccines induce better immunity against pathogenic viruses than isolated viral proteins (see, for example, Putkonen et al., Immunization with Live Attenuated SIVmac Can Protect Macaques against Mucosal Infection with SIVsm, Vaccines 96, pps. 20-210, 1996; Dimmock and Primrose Introduction to Modern Virology, Fourth Ed., Blackwell Science, 1994). The use of live lentivirus vaccines, such as HIV-1 vaccine, is resisted because of great concern that the vaccine virus will persist indefinitely in the inoculated population because of integration of viral DNA into the host DNA of the inoculated individuals (see, for example, Haaft et al., Evidence of Circulating Pathogenic SIV Following Challenge of Macaques Vaccinated with Live Attenuated SIV, Vaccines 96,pps. 219-224, 1996). Thus, a safe and effective vaccine against HIV-1 will encompass modifications to prevent the development of virulent pathogenic infection that could occur by either random mutation or other change to the initially non-pathogenic vaccine virus. This vaccine could be in the form of a live virus vaccine against HIV-1 or a DNA vaccine against HIV-1. DNA vaccines are generally injected into host tissues in the form of plasmid DNA or RNA molecules via needle or particle bombardment. Once delivered, the DNA induces expression of antigenic proteins within transfected cells. U.S. Pat. No. 6,194,389 described methods for transferring DNA to vertebrate cells to produce a physiological immune-response producing protein in the animal subject.

Testing of vaccine efficacy requires inoculation then challenge of the subject with live virus or DNA. It is ethically and practically difficult to attempt preliminary studies using human subjects. The use of model systems for preliminary design and testing of candidate vaccines has been hampered by various species-specific features of the virus. The HIV-1 virus itself is currently known only to infect certain rare and endangered species of chimpanzee in addition to humans. The feasibility of obtaining sufficient numbers of such endangered animals for full preliminary study of HIV-1 virus vaccines is quite low. It is preferable to use validated analogous animal model systems.

One analogous model system for HIV-1 infection has been the SIV_{mac} (Simian Immunodeficiency Virus, macaque) system. Simian Immunodeficiency Virus (SIV) infects a variety of simians, including macaques, but the differences between SIV and HIV make SIV of limited used as a potential human vaccine. There is therefore needed a virus that is closely related to HIV, but still infectious in an animal model, for use in the development of an HIV vaccine.

Deletion of regulatory gene nef from SIV_{mac} rendered the virus avirulent. SIV_{mac}, however, is only an analogous model system for human HIV-1 infection. The system does not replicate all of the salient features of HIV-1 infection of humans (see generally Hu et al., Transmembrane Protein and Core Antigens in Protection against SIV infection, Vaccines 95, pps. 167-173, 1995; Lewis and Johnson, Developing Animal Models for AIDS Research—Progress and Problems, TIBTECH 13:142-150, 1995); Desrosiers, The Simian Immunodeficiency Viruses, Annual Review of Immunology, 8:557-578, 1990).

Chimeric SIV-HIV virus has been developed by placing the envelope proteins of HIV-1 on a background of SIV_{mac}. The Chimeric virus proved to be infectious to monkeys, but did not result in full-blown AIDS or an accurate model to mimic HIV-1 infection in monkeys (see generally Shibata and Adachi, SIV/HIV Recombinants and their use in Studying Biological Properties, AIDS Research and Human Retroviruses 8(3):403-409, 1992; Sakuragi et al., Infection of Macaque Monkeys with a Chimeric Human and Simian Immunodeficiency Virus, J. General Virology, 73:2983-2987, 1992).

As described below, the present invention teaches specific methods, virus constructs, and DNA vaccine constructs that are effective in generating an immune response to HIV-1 in a vaccinated host.

### SUMMARY OF INVENTION

Preferred embodiments of the present invention relate to DNA vaccines for providing an immune response against HIV without exhibiting pathogenicity in the immunized individual because of the disruption of the ability of the DNA molecules to encode for viral proteins critical in producing pathogenicity. Thus, the invention is directed to a vaccine for immunization against HIV comprising an isolated DNA molecule having a sequence encoding a plurality of viral proteins capable of stimulating an immune response against HIV, wherein the combination of said plurality of viral proteins has been rendered nonpathogenic by disrupting the ability of the DNA molecule to encode for at least one of said plurality of viral proteins. The invention further relates to the regulation of the DNA vaccine by the use of its natural promoter, the CMV promoter, or any other suitable promoter.

In one embodiment of the invention, DNA molecules encode proteins of an SIV/HIV Chimeric virus (SHIV) having the LTR, Gag, Pol and Nef of SIV_{mac239} and the Env, Tat, Vpu, and Rev of HIV-1, HXB2C. This DNA molecule can be used as a DNA vaccine to prevent HIV disease in persons at risk for infection, and also for therapeutic immunization of HIV-infected persons on Highly Active Anti-Retroviral Therapy (HAART). The DNA of this Chimeric virus is able to persist in the lymph nodes and induce a cellular immune response against viruses that cause AIDS. Because of this persistence, the vaccine is able to protect against infection and disease caused by pathogenic heterologous viruses that cause AIDS.

The subject matter of the present application also extends to the invention of the parent case of the present application (U.S. patent application Ser. No. 08/850,492), incorporated herein by reference.

### BRIEF DESCRIPTION OF THE DRAWINGS

FIG. 1 is a diagram showing the construction of plasmid-based vaccine pET-9a/ΔvpuΔnefSHIV_{PPC}, also referred to herein as the V3 embodiment of the present invention.

FIG. 2 is a table showing the detection of long-term cytotoxic lymphocytes (CTLs) in macaques immunized with the V3 vaccine.

FIG. 3 is a diagram showing the construction of plasmid-based vaccine pET-9a/ΔrtΔvpuΔnefSHIV_{PPC}, also referred to herein as the V4 embodiment of the present invention.

FIG. 4 is a diagram showing the construction of plasmid-based vaccine pET-9a/ΔrtSHIV_{KU2}, also referred to herein as the V5 embodiment of the present invention.

FIG. 5 is a diagram showing the construction of plasmid-based vaccine pET-9a/ΔrtΔvpuΔnefΔ3′LTR SHIV_{PPC}, also referred to herein as the V4b embodiment of the present invention.

FIG. 6 is a diagram showing the construction of plasmid-based vaccine pET-9a/ΔrtΔ3′LTR SHIVₖᵤ₂, also referred to as the V6 embodiment of the present invention.

FIG. 7 is a diagram showing the construction of the plasmid-based vaccine referred to as the V7 embodiment of the present invention.

FIG. 8 is a schematic diagram showing the schematic layouts of the V5, V6, and V7 embodiments of the present invention, as well as the schematic layout of a vector of the present invention.

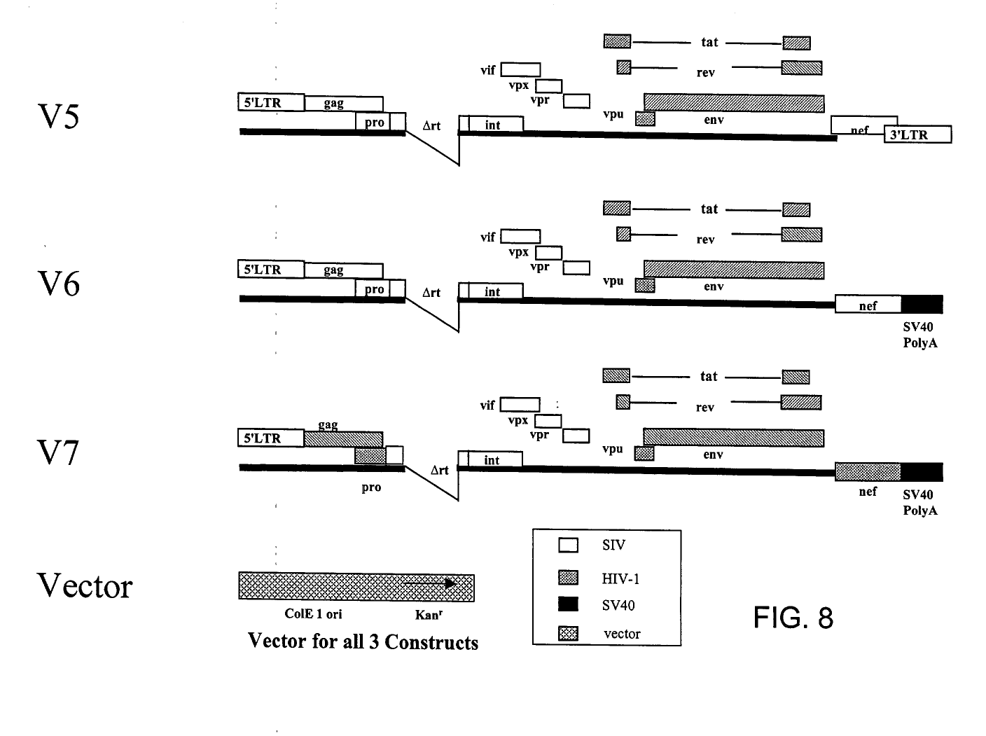

FIG. 9 is a schematic diagram of the V7 DNA construct.

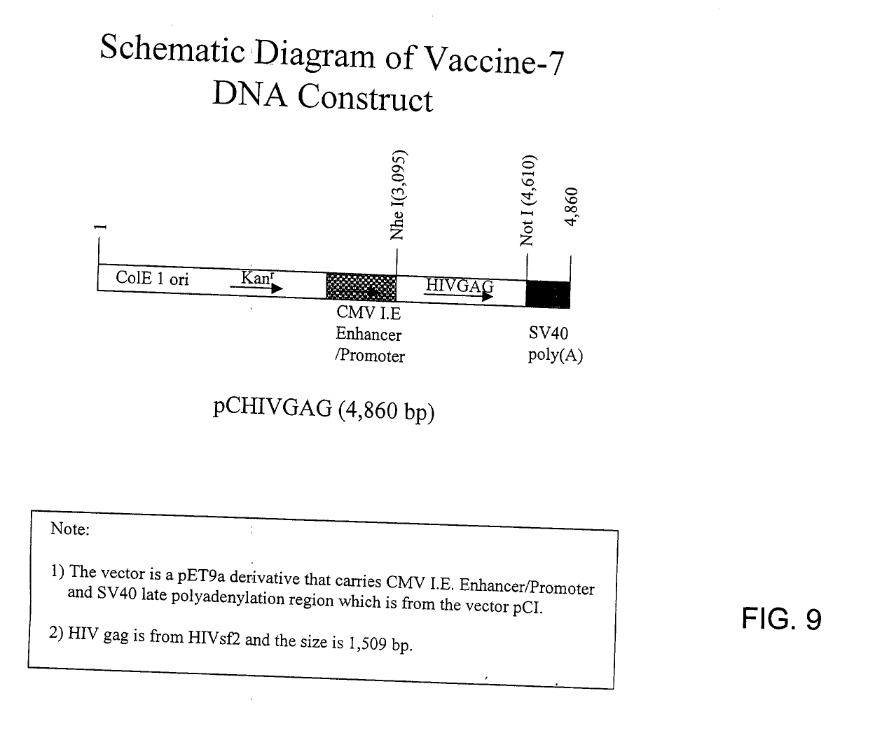

### DETAILED DESCRIPTION OF THE INVENTION

One aspect of the present invention is directed to DNA molecules that encode viral proteins capable of stimulating an immune response against HIV. Importantly, the DNA molecules of the present invention have been disrupted functionally such that the ability to encode proteins that are important in pathogenicity is removed. In another aspect of the present invention, it is preferable that the DNA is disrupted functionally by inserting or deleting at least one nucleotide such that the number of nucleotides in the altered sequence differs with respect to the unaltered sequences. It is most preferable that this differing number of nucleotides with respect to the altered and unaltered DNA apply to the vpu gene. A further aspect of the present invention is the regulation of the DNA molecule by its natural promoter or a CMV promoter, or any other suitable promoter.

A number of DNA sequences are disclosed herein and identified with various SEQ ID NOs. A brief description of each SEQ ID NO is given here for clarity. SEQ ID NO:1 is the nucleotide sequence of the V3 embodiment of the present invention, as described below; SEQ ID NO:2 is the nucleotide sequence of the V4 embodiment of the present invention, as described below; SEQ ID NO:3 is the nucleotide sequence of the V5 embodiment of the present invention, as described below; SEQ ID NO:4 is the nucleotide sequence of the pET-9a vector, as described below; SEQ ID NO:5 is the nucleotide sequence of the V6 embodiment of the present invention, as described below; SEQ ID NO:6 is the nucleotide sequence of the V7 embodiment of the present invention, as described below; SEQ ID NO:7 is the nucleotide sequence of a portion of the vpu gene deleted from some embodiments of the present invention, as described below; SEQ ID NO:8 is the nucleotide sequence of a portion of the nef gene deleted from some embodiments of the present invention, as described below; SEQ ID NO:9 is the nucleotide sequence of a portion of the rt gene deleted from some embodiments of the present invention, as described below; SEQ ID NO:10 is the nucleotide sequence of the overlapping region of the vpu and env genes, as described below; SEQ ID NO:11 is the nucleotide sequence of a portion of the 3′ LTR deleted from some embodiments of the present invention, as described below; and SEQ ID NO:12 is the nucleotide sequence of a portion of the 3′ LTR deleted from other embodiments of the present invention, as described below.

The DNA of a Chimeric virus known as SHIV-4 is comprised of the LTR, gag, pol and nef of SIV_{mac}239, and the env, tat, rev, and vpu of HIV-1, strain HXB2. An alteration of the first codon of the vpu gene of SHIV-4 renders that gene non-functional. SHIV-4 preferentially uses the CXCR4 co-receptor for entry into susceptible cells. It is able to replicate productively in CD4⁺ T cell lines, PBMCs, and macaque PBMCs, but does not replicate productively in either human or macaque macrophages. Experiments performed using co-cultures of inoculated macaque dendritic cells that were later co-cultivated with resting macaque CD4⁺ T cells indicated that SHIV-4 replication in these cells is poor. The SHIV-4 virus is infectious in macaques, but the resulting infection is abortive. Further, the virus induces only limited cellular and humoral immune responses during the transient infection.

Serial passage of the SHIV-4 virus was performed in macaques, eventually yielding a highly pathogenic virus designated SHIV_{KU}. The pathogenesis of SHIV_{KU} has been extensively examined (see, for example, Liu et al., Virology, 260:295-307 (1999); Stephens et al., Virology, 231:313-321 (1997); Foresman et al., AIDS Res. Hum. Retroviruses, 14:1035-1043 (1998); Joag et al., J. Virol., 71:4016-4023 (1997); Joag et al., AIDS Res. Hum. Retroviruses, 15:391-394 (1999); and Joag et al., J. Virol., 72:9069-9078 (1998)). Table 1 provides data for the replication of SHIV_{KU} in human PBMC culture.

TABLE 1
Replication of SHIV_{KU} in Human PBMC Culture (pg/ml of p27)

| | | Control | SHIV_{KU} |
|---|---|---|---|
| Day | 0 | Neg | Neg |
| | 2 | Neg | 525 |
| | 4 | Neg | 12,466 |
| | 6 | Neg | 18,117 |
| | 8 | | 17,988 |
| | 10 | | 15,909 |

The experiment utilized 2×10⁶ PHA stimulated PBMCs. The inoculum was 2000 TCID₅₀ of each virus.

In order to disrupt the pathogenicity of SHIV_{KU}, 60 nucleotides were deleted from the vpu gene, and 217 nucleotides were deleted from the nef gene to create a live virus vaccine known as V1. The V1 virus (Δvpu ΔnefSHIV-4) has the same biological properties in primary macaque cell cultures and inoculated macaques as the parental SHIV-4. It is important to note that the precise deletions detailed above are not the only method of disrupting the vpu and nef genes. Any modification that results in a functional disruption of the genes, including full deletion (or as near a full deletion as possible without disrupting other genes) may be used. The following example details the inoculation of macaques with the V1 vaccine and describes the results of that inoculation.

### EXAMPLE 1

### Immunization of Macaques with V1

Six macaques were immunized with the V1 virus. Six months later, each of these animals was challenged with SHIV_{KU}. In addition, four unvaccinated macaques were also challenged with the virus. All six of the immunized animals became infected with the SHIV_{KU} virus following challenge, but in contrast to the four unvaccinated macaques that succumbed to AIDS within one year following challenge, only one of the six vaccinated macaques developed AIDS during the one year post-challenge period. The other five immunized macaques were able to control replication of the SHIV_{KU} virus with no apparent pathological effects (see Joag et al., J. Virol., 72:9069-9078 (1998)). Thus, the immunization process was deemed a success. During two and three years post-challenge, the SHIV_{KU} virus reappeared in the peripheral blood of four out of five of the surviving immunized macaques, and two of these animals developed AIDS. The other two died of unrelated causes, but had exhibited a resurgence of SHIV_{KU} in PBMCs and it is expected that they would have developed AIDS had death not occurred. Thus, the V1 vaccine was able to cause a delay in the onset of disease, but was not sufficient to provide permanent protection to the immunized macaques.

A new Chimeric virus was cloned from the spleen of a macaque, designated PPc, used in the serial passage of SHIV-4 detailed above. This new chimera was designated SHIV_{PPC}. The env and nef genes of SHIV_{PPC} are closely homologous to the corresponding genes in SHIV_{KU}. Surprisingly, however, SHIV_{PPC} had no pathogenic effect after inoculation into two macaques. The virus became pathogenic after serial passage in macaques (as will be described below). SHIV_{PPC} is similar to SHIV_{KU} in that it is an X4 virus, but it is replication competent in both CD4⁺ T cells and macrophages (although it is less replication competent in macrophages than CD4⁺ T cells). SHIV_{PPC} replicated productively in the two test animals for several weeks, after which the replication was brought under control. Neither of the animals showed any pathological effects, including even a transient loss of CD4⁺ T cells, during the productive phase of the infection.

To render SHIV_{PPC} nonpathogenic, the vpu gene of SHIV_{PPC} was deleted to create ΔvpuSHIV_{PPC}, dubbed the V2 vaccine. The following example details the vaccination of two groups of macaques with this virus to test its efficacy as a vaccine.

### EXAMPLE 2

### Immunization of Macaques with V2

This virus was administered orally to a group of six macaques to test its efficacy as a vaccine. Another group of six macaques received the virus intradermally to test the efficacy of that route of vaccine administration. Undiluted virus stock containing approximately 10³ animal infectious doses (determined by mucosal route titration) was used for the oral inoculation. 100 μg of infectious plasmid DNA was used for the intradermal inoculations. All twelve of the animals became infected by ΔvpuSHIV_{PPC}. The kinetics of the viral replication, as determined by real-time PCR analysis of viral RNA concentrations in plasma, was indistinguishable in the two groups of animals. The plasma viral RNA concentrations reached levels of approximately 10⁵ copies/ml at the height of replication, and productive replication was terminated by 8 weeks post-immunization. All twelve of the macaques developed neutralizing antibodies in plasma and cytotoxic CD8⁺ T cells (CTLs), as well as proliferative antigen-specific CD4⁺ T cells in peripheral blood and lymph nodes, a few months following immunization.

The six animals that were inoculated orally with the V2 virus were monitored for four years. These macaques were challenged vaginally with SHIV_{KU} six months after immunization. All six of the animals became infected with SHIV_{KU}, as indicated by the presence of viral DNA in the lymph nodes (see Joag et al., J. Virol., 72:9069-9078 (1998)). Productive replication of the SHIV_{KU} virus was, however, minimal in all six animals. Biopsies of peripheral lymph nodes from the six animals were performed at approximately six-month intervals, and the tissue was assayed for the presence of DNA from both the vaccine and challenge viruses. In addition, a portion of the lymph nodes were used to obtain cells from which CD8⁺ T cells were depleted. The remaining CD4⁺ T cells were tested for infectivity. The cells were cultivated for virus isolation and the virus isolates were characterized genetically. It was determined that at 18 weeks post-challenge, the DNA of both viruses was present in the lymph nodes of the macaques, but, whereas the DNA of the vaccine virus persisted for the four year duration of the study, the DNA of the challenge virus gradually declined in concentration in these tissues. The DNA of the challenge virus had become undetectable in four of the six animals by 24 months post-challenge. Of the two animals that did have challenge virus DNA, lymph node cells from one of them yielded SHIV_{KU} (see Silverstein et al., J. Virol., 74:10489-10497 (2000)). These results indicated that, while the V2 vaccine was unable to prevent infection by the challenge virus, it did prevent disease and, remarkably, contributed to the eventual elimination of the challenge virus and its DNA from the lymph nodes. This study, then provided new and unexpected results: namely, that although the DNA of the challenge virus was clearly detectable in the lymph nodes, concentrations of this DNA became progressively less with time. This indicates that infection by the challenge virus can be cured by the use of vaccines prepared in accordance with the teachings of the present invention.

In order to determine whether prior immunization of macaques with the V1 vaccine would induce sufficient immunity to attenuate the replication of the V2 vaccine (thereby making it safer), yet still allow the animals to acquire the protective immunity induced by the V2 vaccine, seven macaques were immunized with the V1 vaccine and, four months later, administered the V2 vaccine orally. None of these animals developed productive infection in PBMCs following inoculation with the V2 vaccine. A second inoculation with the V2 vaccine was performed three months later, by subcutaneous injection, to ensure that the animals had indeed been infected with the V2 virus. Again, there was no evidence of productive replication of the V2 virus.

Next, the animals were challenged by rectal infusion of three pathogenic viruses, SHIV_{KU}, SHIV 89.6P (see Reimann et al., J. Virol, 70:6922-6928 (1996)), and a neurovirulent strain of SIV_{mac} known as SIV 17E (see Sharma et al., J. Virol., 66:3550-3556 (1992)), approximately one year after the last immunization. The immune response of the animals to each of these viruses was assessed prior to the challenge. Each of the challenged animals developed neutralizing antibodies to the vaccine viruses, and less to the challenge viruses. None of the animals developed neutralizing antibodies to SIV 17E. The animals were then assessed for CTL responses using CD4⁺ T cells immortalized with Herpesvirus saimiri for infection with the three pathogenic viruses, respectively, and B cells transformed with Herpesvirus papio for infection with recombinant vaccinia viruses expressing HIV Env, SIV Gag, and SIV Pol. Prior to the challenge, each of the seven macaques had CTLs against each of the three challenge viruses. Using the B cell assay system, it was determined that they had CTLs to SIV Pol, SIV Gag, and HIV Env, but not to SIV Env. All three of the challenge viruses caused infection in each of the seven animals, but, similar to the animals described above that were immunized with ΔvpuSHIV_{PPC} (V2), all seven of the animals controlled productive infection with each of the three agents. Long-term study of the seven animals over the course of the following 13 months demonstrated that the challenge viruses had the same fate as the SHIV_{KU} used to challenge the animals immunized with the V2 virus alone. The three-virus cocktail challenge resulted in a massive anamnestic boost of the cell-mediated immune responses and the increased level of activity persisted indefinitely. None of the seven animals developed any sign of disease. At 82 weeks post-challenge, an examination of the lymph nodes of the seven animals showed that only one had SIV DNA, while two had SHIV 89.6 DNA and four had SHIV_{KU} DNA. Infectious virus was isolated from CD8-depleted lymph node cells from two of the four animals. One had SHIV_{KU} and SHIV 89.6 DNA, the other had SHIV_{KU} and SIV DNA.

The preceding results indicate that the protective immunity provided by V2, characterized by the elimination of challenge viruses as well as their corresponding DNAs, could be boosted by administering both vaccine and challenge viruses in the absence of productive replication of the boosting agents. Unexpectedly, SIV, which is the most heterologous of the three challenge viruses, was eliminated with the greatest efficiency. The fact that the vaccine provided protection against heterologous as well as homologous pathogenic viruses is important in demonstrating the efficacy of the vaccines of the present invention.

In order to further ensure the safety of the vaccine, an additional deletion in the nef gene was made in the ΔvpuSHIV_{PPC}. The result was the ΔvpuΔnefSHIV_{PPC} virus (the V3 vaccine).

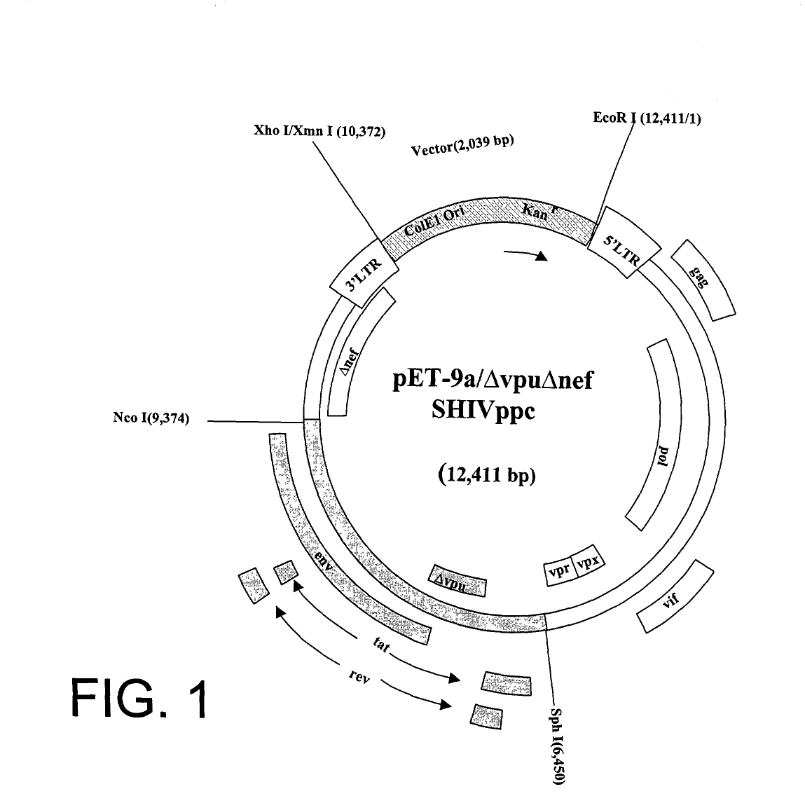

A schematic diagram of the V3 vaccine DNA in vector pET-9a is provided in FIG. 1. In FIG. 1, the cross-hatched portion of the diagram indicates the vector, the gray portions of the diagram indicate material taken from HIV, and the uncolored portions of the diagram indicate material taken from SIV. The 2.3 kb EcoRI/XmnI fragment of the pET-9a plasmid is replaced by the 10 kb provirus genome of the vaccine. An EcoRI restriction site was created immediately upstream of the 5′ LTR and an XhoI restriction site was created immediately at the end of the 3′ LTR. As can be seen from the schematic, the vpu and nef genes were permanently disrupted. This was accomplished by deletion of 62 bp and 216 bp in these genes, respectively. The sequence of the V3 vaccine DNA in vector pET-9a is provided in SEQ ID NO:1. The precise deletions made in the vpu and nef are provided in Table 2.

TABLE 2

Deleted Sequences in V3 Vaccine

| | |
|---|---|
| Gene | Sequence Deleted |

| | |
|---|---|
| vpu | 5′- |
| | ATATTAAGAC AAAGAAAAAT |
| | AGACAGGTTA ATTGATAGAC |
| | TAATAGAAAG AGCAGAAGAC |
| | AG |
| | -3′ |
| nef | 5′- |
| | CGACCTACAA TATGGGTGGA |
| | GCTATTTCCA TGAGACGGTC |
| | CAGGCCGTCT GGAGATCTGC |
| | GACAGAGACT CTTGCGGGCG |
| | TGTGGGGAGA CTTATGGGAG |
| | ACTCTTAGGA GAGGTGGAAG |
| | ATGGATACTC GCAATCCCCA |
| | GGAGGATTAG ACAAGGGCTT |
| | GAGCTCACTC TCTTGTGAGG |
| | GACAGAAATA CAATCAGGAA |
| | CAGTATATGA ATACTC |
| | -3′ |

In addition, the precise sequence deleted from the vpu gene is that sequence provided in SEQ ID NO:7. The precise sequence deleted from the nef gene is that sequence provided in SEQ ID NO:8. The precise location of these sequences within the vpu and nef genes can be readily determined as the gene sequences are known in the art and the location of the deleted sequences can be determined manually or via any computer program designed to align DNA sequences. It is understood, of course, that any modifications to the vpu and nef genes sufficient to disrupt their functionality are acceptable. The disruption of the genes may even include full deletion of the genes, with the exception of a portion of the vpu gene sequence that overlaps with the env gene sequence. The overlapping sequence is shown in Table 3.

TABLE 3
Overlapping Sequence in vpu and env Genes

5′-
ATGAGAGTGA AGGAGAAATA TCAGCACTTG TGGAGATGGG
GGTGGAGATG GGGCACCATG CTCCTTGGGA TGTTGATGAT
CTGTAG-3′

The sequence of the vpu and env genes that overlaps is that sequence provided in SEQ ID NO:10. The size of the V3 vaccine DNA construct is 12,411 bp, with 2039 bp comprising the vector and 10,372 bp comprising the provirus genome. In order to determine whether the deletions would cause a reduction in infectivity or in the immunity induced by the virus or its infectious DNA, the experiment detailed in the following example was performed.

### EXAMPLE 3

### Immunization of Macaques with V3

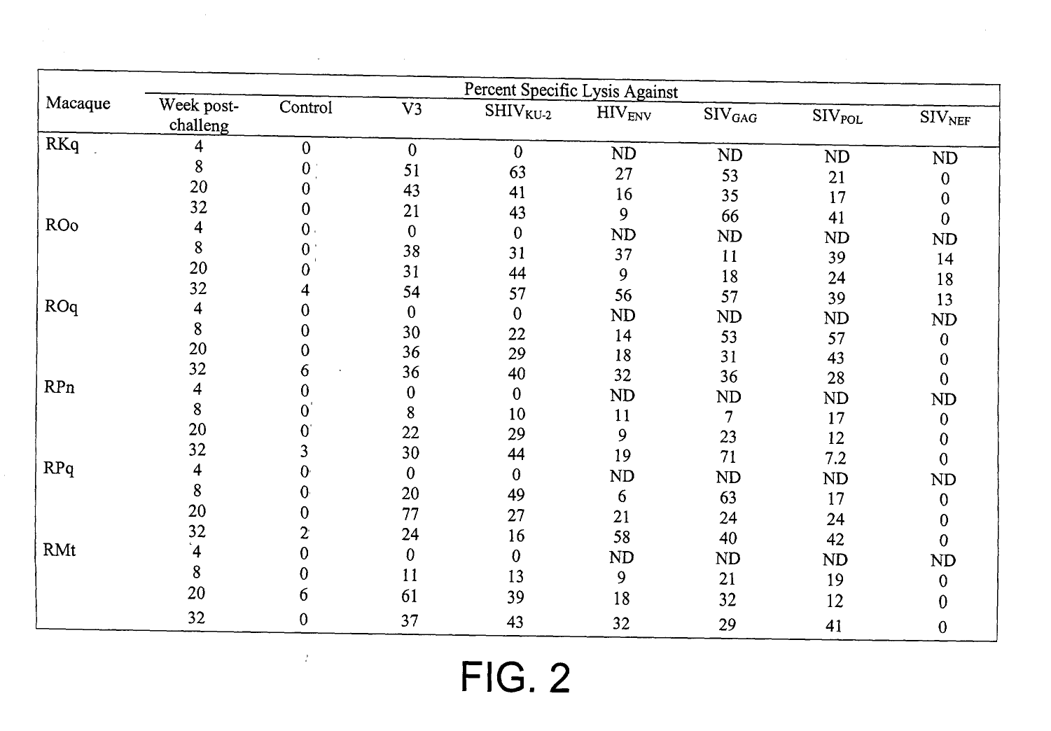

Six macaques were inoculated orally with the V3 virus. Two other macaques were inoculated intracerebrally with 100 μg of V3 infectious plasmid DNA (see FIG. 1). All eight of these animals became infected by the V3 virus. A comparison of plasma viral RNA burdens demonstrated that the concentrations and duration in plasma were equivalent among the animals as well as to the values obtained in animals that had been immunized with the V2 virus described above. The cell-mediated immune responses were also equivalent except that the animals immunized with the V3 virus did not develop CTLs against Nef, as the nef gene had been deleted from the V3 virus (FIG. 2 provides data from the detection of long-term CTLs in macaques immunized with the V3 virus). Thus, it was determined that the V3 and V2 viruses were equivalent in their potential to induce protective immunity.

In order to confirm that the V3 virus would not become pathogenic during passage in macaques, and that the vpu and nef genes were indeed important for the development of pathogenic variants, the following experiment was performed.

### EXAMPLE 4

### The Safety of the V3 Vaccine

Serial inoculations of macaques were performed with the V3 virus and with the parental SHIV_{PPC} virus described above. A macaque was inoculated intravenously with each virus. Three to four weeks later, 3 ml of heparinized blood from the animal was inoculated into two new recipient macaques. This procedure was repeated for a total of four passages for each virus. The viruses replicated productively in each of the eight animals used in the study. The replication pattern of the V3 virus was constant in each passage, and the productive infection was brought under control in all four animals. None of these animals lost CD4⁺ T cells, and all four remained healthy six months following the last passage. The SHIV_{PPC} virus replicated more productively than the V3 virus and the productive infection persisted. The animal in the first passage, however, remained healthy. This changed in subsequent passages, with animals in passages two, three and four all developing the loss of CD4⁺ T cells. This loss became more severe with each passage. Similar to the observations described above with respect to the SHIV_{KU} virus, the SHIV_{PPC} virus from passage four had several mutations in the nef gene that suggested enhanced transcriptional efficiency of the viral DNA. These experiments confirmed the importance of the vpu and nef genes in pathogenesis, and also confirmed that the additional deletion of the nef gene from the V2 virus (to form the V3 virus) did not compromise the immunogenicity and, by inference, the efficacy of the vaccine.

In accordance with the present invention, an infectious DNA vaccine was derived from the V3 virus. To render the DNA molecule safer, additional deletions were made, including the deletion of the gene encoding the reverse transcriptase (RT) enzyme. The deletion of the reverse transcriptase gene eliminates the ability of the virus coded for by the DNA to make a DNA copy of its RNA genome. The resulting DNA molecule was ΔrtΔvpuΔnef SHIV_{PPC} (the V4 vaccine).

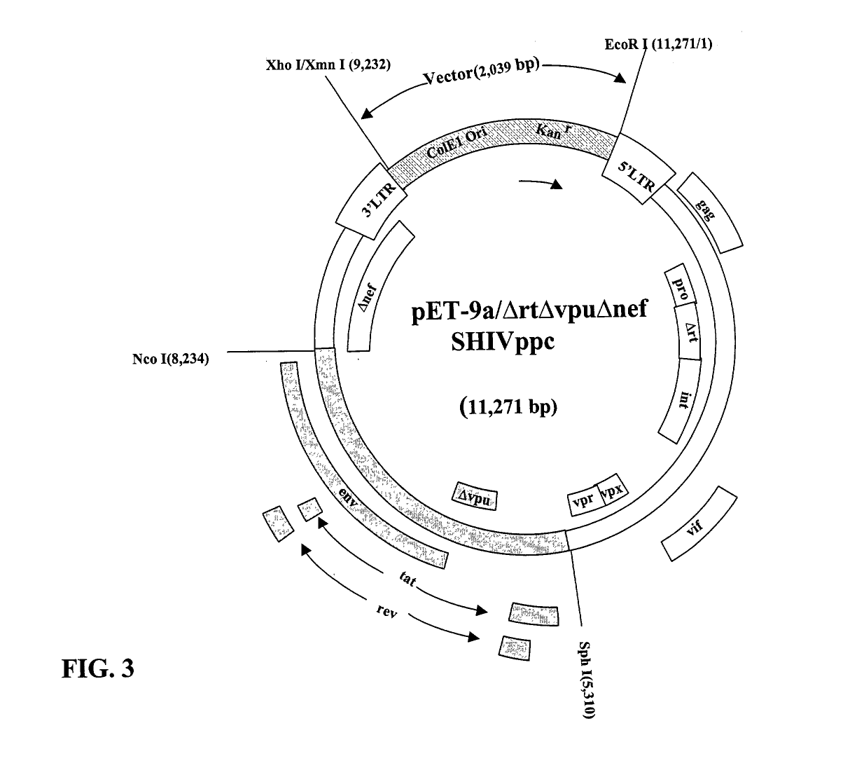

A schematic diagram of the V4 vaccine DNA in vector pET-9a is provided in FIG. 3. The DNA is derived from a virus developed by the inventor of the present vaccine and shown to induce long-term cellular immune responses associated with curative immunity against homologous and heterologous challenge viruses. The vector for this embodiment is pET-9a. The 2.3 kb EcoRI/XmnI fragment of this plasmid was replaced by the 9 kilobase provirus genome of the vaccine. An EcoRI restriction site was created immediately upstream of the 5′ LTR and an XhoI restriction site was created immediately at the end of the 3′ LTR. The sequence of the V4 vaccine DNA is provided in SEQ ID NO:2. The vpu and nef genes were disrupted by the deletion of 62 base pairs and 216 base pairs, respectively. The precise sequences deleted were the same as those outlined with respect to the V3 vaccine in Table 3, above. The rt sequence was disrupted by deletion of 1137 base pairs while the protease and integrase genes were kept intact. The deletion of 1137 base pairs from the rt gene represents a virtually total elimination of the gene. The precise deletion sequence is not provided here because any 1137 base pair deletion that leaves the protease and integrase genes intact is acceptable. The sequence of the rt gene, as well as those of the protease and integrase genes, are well-known in the art. The size of the construct is 11,274 base pairs, being composed of a 2033 base pair vector and the 9241 base pair provirus genome. The following examples detail experimental studies performed with the V4 DNA vaccine.

### EXAMPLE 5

### Transfection of V4 DNA into CEM 174 Cells

Five μg of V4 DNA was transfected into approximately 2×10⁶ CEM 174 cells. The transfected cell cultures developed fusion CPE on day two following transfection. Supernatant fluid was collected from the culture at two-day intervals and the viral p27 content of the supernatant fluid was assessed. After each collection of supernatant fluid, the cell cultures were washed and placed in fresh medium to ensure that each two-day sample contained only viral p27 produced during the preceding two-day period. Approximately 500 pg of p27 was detected in the supernatant fluid on day two. Days four and six also yielded 500 pg of viral p27. On day eight only 250 pg of viral p27 was detected, and by day ten the sample was negative for viral p27. It was determined that most of the viral p27 was cell-associated, with only a small amount appearing in the supernatant fluid. The apparent failure of the cells transfected with V4 DNA to shed viral proteins from their surfaces suggests that the transfected cells would be the only ones capable of presenting viral antigens for induction of an immune response. Though the transfection was a success it remained desirable to produce a DNA that would cause viral proteins to be shed into the extracellular environment.

### EXAMPLE 6

### The Safety and Efficacy of V4

Portions of the V4 supernatant fluid containing viral p27 and described above were inoculated into fresh cultures of CEM 174 cells. These new CEM 174 cells did not develop CPE and the supernatant fluids from these cultures lacked the molecules necessary to code for infectious virus particles. Thus, it was determined that the V4 DNA is safe and is unable to produce infectious viral particles.

In order to determine whether the proteins expressed by the V4 vaccine virus would indeed be recognized by antibodies from an HIV-infected person, as well as by antibodies from previously immunized macaques, cell cultures infected with the vaccine virus and HIV-1, respectively, were pulsed with ³⁵S-labelled methionine, and then lysed and immunoprecipitated with serum from a long-term non-progressor with HIV infection as well as with serum from a macaque that had been previously immunized with the V3 vaccine virus. Both of these sera bound the Env and Gag of both HIV and SIV in the infected cultures. One can infer from these results that the SIV Gag encoded by the vaccine DNA will induce immune responses in immunized humans that will cross-react with HIV Gag. This response would be expected to be boosted following exposure of the immunized individual to HIV.

### EXAMPLE 7

### Inoculation of Macaques with V4 DNA

Four macaques were injected intradermally (ID) with 1 mg of V4 DNA each. Each of three aliquots of approximately 300 μg of DNA were injected ID in both axillae and in one of the inguinal areas of each of the four animals. The other inguinal area of each animal was injected with saline. Examination of blood samples collected from the macaques on a weekly basis showed no evidence of infection in the PBMCs (i.e. no viral DNA as determined by real time PCR). One month after immunization with V4 DNA, two lymph nodes were biopsied from each animal. One biopsy was taken from one of the sites that was injected with vaccine and the other was taken from the inguinal site that received saline. Quantitation of the vaccine DNA by real time PCR showed that the injected sites had between 10 and 30 copies of viral DNA per μg of tissue DNA. Of the four sites that were not injected with the DNA, three of the lymph nodes had undetectable vaccine DNA, and the fourth had one copy. Thus, despite the minute amount injected, the DNA was still able to persist. Importantly, the DNA did not spread. At the four-week time point, two of the animals were injected again with 1 mg of V4 DNA and, two weeks later, lymph nodes were again obtained from all four animals. The lymph nodes from the two animals that had received a single inoculation had approximately 5 copies/μg of lymph node DNA, while the lymph nodes from the animals that were reinjected had 126 and 64 copies of the vaccine DNA/μg tissue DNA, respectively. Thus, the reinjection of DNA had an additive effect on the DNA concentrations in the lymph nodes. This is an important finding because persistence of the DNA in the live virus vaccines was associated with the success of the vaccine in eliminating challenge viruses. The finding that the V4 DNA persisted, and that the amount of DNA could be supplemented by further injection of V4 DNA, supports the conclusion that the V4 DNA vaccine behaves like the DNA of its replication-competent parental virus. Further, these results demonstrate that the long-term persistence of the vaccine DNA that was achieved by replication of the live virus could be achieved by supplemental injections of the non-replicating vaccine DNA at appropriate time intervals.

### EXAMPLE 8

### Long Term Results with V4 and Macaques

Ten weeks after the most recent DNA injection, all four of the macaques described above retained traces of vaccine DNA in their lymph nodes. In addition, all four animals retained effector anti-viral cytotoxic T-lymphocytes in the circulating blood. Thus, the V4 DNA has induced long-lasting cellular immune responses to the virus.

SHIV_{KU} was utilized to develop a DNA vaccine that provides transfected cells with the ability to shed viral proteins into the extracellular environment while retaining a safety and efficacy at least equal to that of the V4 DNA vaccine. SHIV_{KU} was used because the molecular clone of SHIV_{KU} developed by the inventor of the present invention was shown to be highly efficient in replication in macaques and human PBMC cultures. In addition, SHIV_{KU} has a high degree of pathogenicity in macaques. Rapid replication of the virus causes subtotal elimination of the CD4⁺ T cell population within a few weeks of infection (as described above). In addition, when administered to animals that have been previously immunized with vaccine viruses, SHIV_{KU} induces a potent ananmestic immune response that is associated with the development of curative immunity against the virus (see Silverstein et al., J. Virol., 74:10489-10497 (2000)). The high replicative efficiency of the virus was found to be associated with enhanced transcription of viral RNA, which in turn appears to be mediated by a unique interaction between Nef, the transcription factor NFAT, and sequences in the U3 region of the viral promoter. Further, as detailed elsewhere above, the DNA of SHIV_{KU} exhibited better persistence in the lymph nodes of challenged animals than did the DNA of SHIV 89.6P and SIV. The ability of this DNA to persist in the lymph nodes, in addition to its enhanced capacity for expressing viral proteins, are major assets in the efficacy of the DNA as a DNA vaccine. In order to render this embodiment of the present invention safe, the sequences encoding reverse transcriptase were removed, resulting in the ΔrtSHIV_{KU2} DNA vaccine (the V5 vaccine).

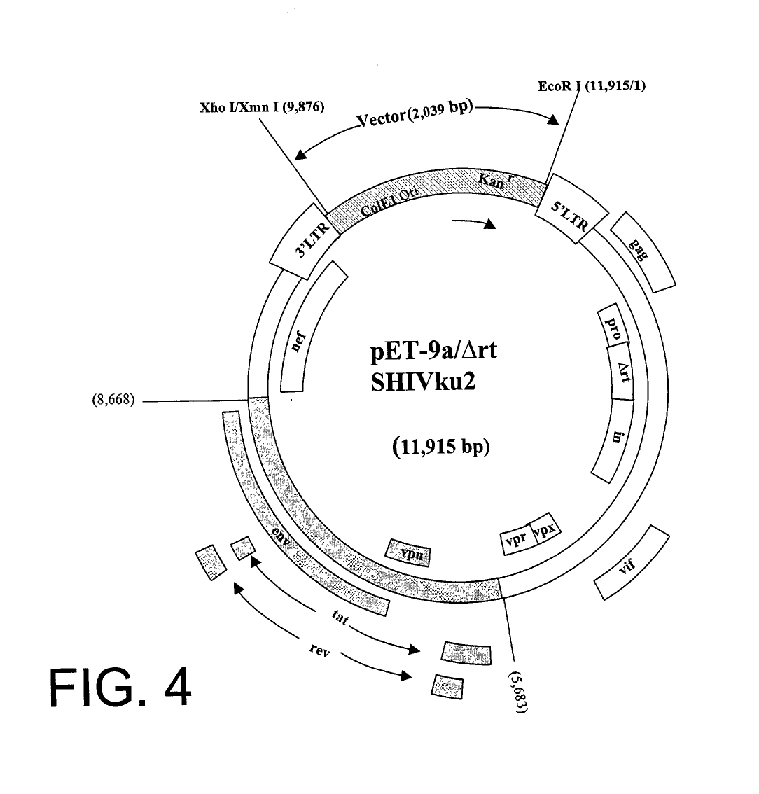

A schematic diagram of the V5 vaccine DNA construct is provided in FIG. 4. As can be seen in FIG. 4, the vector used for this embodiment of the present vaccine is pET-9a. The 2.3 kb EcoRI/XmnI fragment of the plasmid was replaced by the 9.88 kilobase provirus genome of SHIV_{KU2}. An EcoRI restriction site was created immediately upstream of the 5′ LTR and an XhoI restriction site was created immediately at the end of the 3′0 LTR. The sequence of the V5 DNA vaccine is provided in SEQ ID NO:3. The rt sequence was disrupted by deletion of 762 base pairs, while the protease and integrase genes were left intact. The precise deletions made in the rt are provided in Table 4.

TABLE 4

Deleted rt Sequence in V5 Vaccine

| | |
|---|---|
| Gene | Sequence Deleted |

| | |
|---|---|
| rt | 5′- |
| | AGCCATCTTC CAATACACTA |
| | TGAGACATGT GCTAGAACCC |
| | TTCAGGAAGG CAAATCCAGA |
| | TGTGACCTTA GTCCAGTATA |
| | TGGATGACAT CTTAATAGCT |
| | AGTGACAGGA CAGACCTGGA |
| | ACATGACAGG GTAGTTTTAC |
| | AGTCAAAGGA ACTCTTGAAT |
| | AGCATAGGGT TTTCTACCCC |
| | AGAAGAGAAA TTCCAAAAAG |
| | ATCCCCCATT TCAATGGATG |
| | GGGTACCAAT TGTGGCCAAC |
| | AAAATGGAAG TTGCAAAAGA |
| | TAGAGTTGCC ACAAAGAGAG |
| | ACCTGGACAG TGAATGATAT |
| | ACAGAAGTTA GTAGGAGTAT |
| | TAAATTGGGC AGCTCAAATT |
| | TATCCAGGTA TAAAAACCAA |
| | ACATCTCTGT AGGTTAATTA |
| | GAGGAAAAAT GACTCTAACA |
| | GAGGAAGTTC AGTGGACTGA |
| | GATGGCAGAA GCAGAATATG |
| | AGGAAAATAA AATAATTCTC |
| | AGTCAGGAAC AAGAAGGATG |
| | TTATTACCAA GAAGGCAAGC |
| | CATTAGAAGC CACGGTAATA |
| | AAGAGTCAGG ACAATCAGTG |
| | GTCTTATAAA ATTCACCAAG |
| | AAGACAAAAT ACTGAAAGTA |
| | GGAAAATTTG CAAAGATAAA |
| | GAATACACAT ACCAATGGAG |
| | TGAGACTATT AGCACATGTA |
| | ATACAGAAAA TAGGAAAGGA |
| | AGCAATAGTG ATCTGGGGAC |
| | AGGTCCCAAA ATTCCACTTA |
| | CCAGTTGAGA AGGATGTATG |
| | GGAACAGTGG TGGACAGACT |
| | ATTGGCAGGT AACCTGGATA |
| | CC |
| | -3′ |

The precise sequence deleted is that sequence provided in SEQ ID NO:9. The precise location of the sequence in Table 4 within the rt gene can be readily determined as the rt gene sequence is known and the location of the deleted sequence can be determined manually to via any computer program designed to align DNA sequences. It is understood, of course, that any modification to the rt gene sufficient to disrupt its functionality is acceptable. The disruption of the gene may even include a full deletion of the rt gene. The size of the construct is 11,915 base pairs, being composed of a 2033 base pair vector and the 9882 base pair provirus genome. The following examples detail experimental studies performed with the V5 vaccine.

### EXAMPLE 9

### Transfection of V5 DNA into CEM 174 Cells

Five μg of V5 DNA was transfected into approximately 2×10⁶ CEM 174 cells. The transfected cell cultures developed fusion CPE on day four following transfection. Supernatant fluid was collected from the culture at two-day intervals and the viral p27 content of the supernatant fluid was assessed. After each collection of supernatant fluid, the cell cultures were washed and placed in fresh medium to ensure that each two-day sample contained only viral p27 produced during the preceding two-day period. Approximately 3050 pg of viral p27 was detected in the supernatant fluid on day four. As was the case with the V4 cultures described above, the V5 cultures became negative by day ten. Decline in viral protein production coincided with the disappearance of the syncytial cells from each culture, presumably by apoptotic mechanisms because the cell culture system utilized is highly susceptible to viral-induced fusion CPE. Importantly, most of the viral p27 observed was located in the supernatant fluid. The ability of the V5 transfected cells to shed viral proteins into the extracellular environment provides an opportunity for other cells to present viral antigens. Therefore, in addition to the ability of V5 DNA to cause enhanced transcription of its RNA and produce more viral proteins, the ability to shed viral proteins into the extracellular environment provides an added advantage.

### EXAMPLE 10

### The Safety and Efficacy of V5

Portions of the V5 supernatant fluid containing viral p27 and described above were inoculated into fresh cultures of CEM 174 cells. These new CEM 174 cells did not develop CPE and the supernatant fluids from these cultures lacked the moleculaes necessary to code for infectious viral particles. Thus, it was determined that the V5 embodiment of the present invention is safe and is unable to produce infectious viral particles.

In order to determine whether the proteins expressed by the V5 vaccine virus would indeed be recognized by antibodies from an HIV-infected person, as well as by antibodies from previously immunized macaques, cell cultures infected with the vaccine virus and HIV-1, respectively, were pulsed with ³⁵S-labelled methionine, and then lysed and immunoprecipitated with serum from a long-term non-progressor with HIV infection as well as with serum from a macaque that had been previously immunized with the V3 vaccine virus. Both of these sera bound the Env and Gag of both HIV and SIV in the infected cultures.

The 3′ and 5′ long-terminal repeats (LTR) of HIV are necessary for proper integration of the virus into the host genome. In addition, the 5′ LTR contains the natural viral promoter and thus, in the present vaccine, is necessary for expression of viral genes. Eliminating the 3′LTR provides a virus that is unable to integrate into the host genome, while retaining the ability to encode for immunogenic viral proteins without encoding for infectious virus. This decreases the likelihood that the vaccine DNA will become inserted into a host oncogene, thereby causing oncogenesis. Thus, two additional embodiments of the present vaccine, known as the V4B and V6 embodiments, were created.

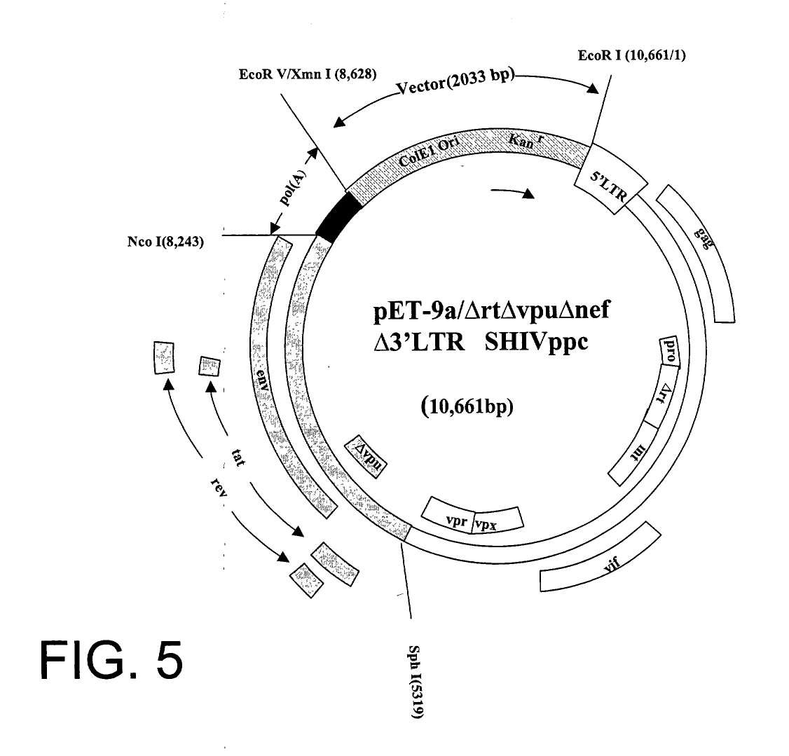

A schematic diagram of the pET-9a/ΔrtΔvpuΔnefΔ3′LTR SHIV_{PPC} (V4B) vaccine DNA construct is provided in FIG. 5. The V4B vaccine represents an alternative embodiment of the present invention. As can be seen in FIG. 5, the vector used for this embodiment of the present vaccine is pET-9a. The 2.3 kb EcoRI/XmnI fragment of the plasmid was replaced by the 8.24 kb provirus genome and 385 bp SV40 polyadenylation sequences. An EcoRI restriction site was created immediately upstream of the 5′ LTR, and an EcoRV site was created immediately at the end of the SV40 polyadenylation sequences. The sequence of the V4B embodiment of the present invention is the same as the sequence of the V4 embodiment, provided in SEQ ID NO:2, with an additional modification as described below. The vpu gene was permanently eliminated by a 62 bp deletion that included the initiation codon. The 62 bp sequence deleted is the same as that deleted with respect to the V3 embodiment of the present invention and provided in Table 2, above. The rt gene was eliminated by the deletion of 1137 bp, while the genes coding for viral protease and integrase were left intact. The deletion of 1137 base pairs from the rt gene represents virtually a total elimination of the gene. The precise deletion sequence is not provided here because any 1137 base pair deletion that leaves the protease and integrase genes intact is acceptable. The sequence of the rt genes, as well as those of the protease and integrase genes, are known in the art. The nef gene and the 3′ LTR were deleted by inserting the SV40 polyadenylation sequences between the end of the env gene and the vector. Because the location and sequence of the env gene is known in the art, it is readily apparent how the deletion of nef and the 3′ LTR was accomplished based on the disclosure above.

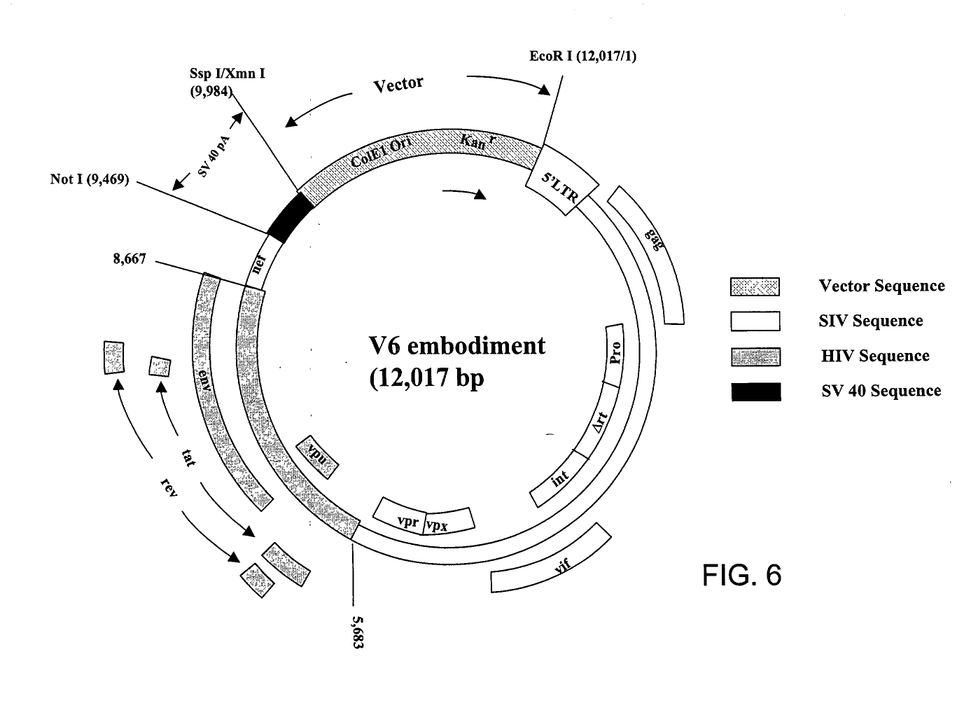

A schematic diagram of the pET-9a/ΔrtΔ3′LTR SHIVₖᵤ₂ (V6) vaccine DNA construct is provided in FIG. 6. The V6 vaccine represents an alternative embodiment of the present invention. The sequence of the V6 embodiment of the present invention is provided in SEQ ID NO:5. As can be seen in FIG. 6, the vector used for this embodiment of the present vaccine is pET-9a. The 2.3 kb EcoRI/XmnI fragment of the plasmid was replaced by the SHIVₖᵤ₂ provirus genome and a 515 bp SV40 polyadenylation sequences. An EcoRI restriction site was created immediately upstream of the 5′ LTR, and SV 40 polyadenylation sequences were added to the end of the nef gene. The rt gene was eliminated by the deletion of a 762 bp sequence, while the genes coding for viral protease and integrase were left intact. The precise 762 bp sequence deleted from the rt gene is the same as that deleted in the V5 embodiment of the present invention as provided in Table 4, above. The 3′ LTR was also disrupted, but only through a partial deletion due to the overlap of the 3′ LTR with the nef gene. The precise sequence of bases deleted from the 3′ LTR is provided in Table 5. Although FIG. 6 shows the V6 embodiment of the present invention as having an SIV nef gene, it is contemplated that the vaccine could alternatively have a nef gene derived from HIV.

TABLE 5
Sequence Deleted from 3′ LTR of V6 Embodiment

5′-
AACAGCAGGG ACTTTCCACA AGGGGATGTT ACGGGGAGGT
ACTGGGGAGG AGCCGGTCGG GAACGCCCAC TTTCTTGATG
TATAAATATC ACTGCATTTC GCTCTGTATT CAGTCGCTCT
GCGGAGAGGC TGGCAGGTTG AGCCCTGGGA GGTTCTCTCC
AGCACTAGCA GGTAGAGCCT GGGTGTTCCC TGCTAGACTC
TCACCAGCAC TTGGCCGGTG CTGGGCAGAG TGATTCCACG
CTTGCTTGCT TAAAGCCCTC TTCAATAAAG CTGCCATTTT
AGAAGTAAGC TAGTGTGTGT TCCCATCTCT CCTAGCCGCC
GCCTGGTCAA CTCGGTACTC AATAATAAGA AGACCCTGGT
CTGTTAGGAC CCTTTCTGCT TTGGGAAACC GAAGCAGGAA
AATCCCTAGC A
-3′

The precise DNA sequence provided in Table 5 is the same as that provided in SEQ ID NO:11.

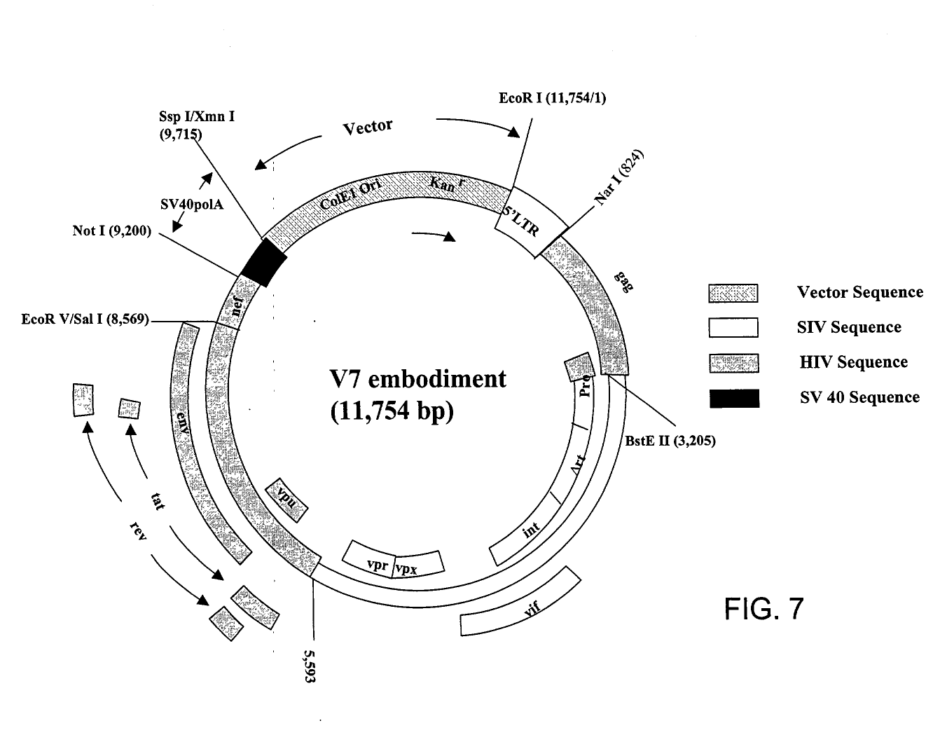

Another embodiment of the vaccine of the present invention is designated as the V7 embodiment. The sequence of the V7 embodiment of the present invention is provided in SEQ ID NO:6. A schematic diagram of the V7 embodiment of the present invention is provided in FIG. 7. The vector used is pET-9a. The 2.3 kb EcorI/Xmn I fragment of the plasmid was replaced by the SHIVₖᵤ₂ provirus genome and SV 40 polyadenylation sequences. The rt gene was disrupted by deletion of a 818 bp sequence, while the protease and integrase genes were kept intact. The precise 818 bp sequence deleted from the rt gene is the same as that deleted in the V5 embodiment of the present invention provided in Table 4, above. The sequence of the deleted 3′ LTR of the V7 embodiment is provided in Table 6.

TABLE 6
Sequence Deleted from 3′ LTR of V7 Embodiment

5′-
TGGAAGGGAT CTTTTACAGT GCAAGAAGAC ATAGAATCTT
AGACATGTAC TTAGAAAAGG AAAAAGGCAT CATACCAGAT
TGGCAGGATT ACACCTCAGG ACCAGGAATT AGATACCCAA
AGACATTTGG CTGGCTATGG AAATTAGTCC CTGTAAATGT
ATCAGATGAG GCACAGGAGG ATGAAGAGCA TTATTTAATG
CATCCAGCTC AAACTTCCCA GTGGGATGAC CCTTGGAGAG
AGGTTCTAGC ATGGAAGTTT GATCCAACTC TGGCCTACAC
TTATGAGGCA TATGTTAGAT ACCCAGAAGA GTTTGGAAGC
AAGTCAGGCC TGTCAGAGGA AGAGGTTAAA AGAAGGCTAA
CCGCAAGAGG CCTTCTTAAC ATGGCTGACA AGAAGGAAAC
TCGCTGAAAC AGCAGGGACT TTCCACAAGG GGATGTTACG
GGGAGGTACT GGGGAGGAGC CGGTCGGGAA CGCCCACTTT
CTTGATGTAT AAATATCACT GCATTTCGCT CTGTATTCAG
TCGCTCTGCG GAGAGGCTGG CAGGTTGAGC CCTGGGAGGT
TCTCTCCAGC ACTAGCAGGT AGAGCCTGGG TGTTCCCTGC
TAGACTCTCA CCAGCACTTG GCCGGTGCTG GGCAGAGTGA
TTCCACGCTT GCTTGCTTAA AGCCCTCTTC AATAAAGCTG
CCATTTTAGA AGTAAGCTAG TGTGTGTTCC CATCTCTCCT
AGCCGCCGCC TGGTCAACTC GGTACTCAAT AATAAGAAGA
CCCTGGTCTG TTAGGACCCT TTCTGCTTTG GGAAACCGAA
GCAGGAAAAT CCCTAGCA -3′

The precise sequence provided in Table 6 is the same as that provided in SEQ ID NO:12.

One significant aspect of the DNA embodiments of the present invention lies in the fact that infectious DNAs encoding viruses developed and characterized by the inventor of the present vaccine were constructed, and the nucleotide sequences encoding the reverse transcriptase (RT) protein of the viruses were deleted. Thus, the DNA molecule of the present invention produces viral particles within the host cells, but such viral particles are non-pathogenic. These viral particles are, however, processed by antigen-presenting cells of the immune system, leading to the development of an antiviral immune response. Further, the infected cell can produce these viral particles indefinitely, providing long-term antiviral protection. Thus, an advantage of the present vaccine is that the DNA behaves similarly to DNA of the pathogenic virus, except that it is non-pathogenic because of its lack of the RT coding sequences. The present vaccine is therefore safe.

The fact that vaccine DNA was found in biopsied lymph nodes after only 300 μg of the DNA had been inoculated into the area of the chain of lymph nodes, and the fact that the inoculation had been performed six weeks prior to the biopsy, indicates that the V4 DNA vaccine is behaving like the DNA of its parental, replication-competent virus. Data indicates that V5 may even be a better vaccine than V4. Further, the fact that either minimal or no plasmid DNA was found in lymph nodes at sites distant from the injection sites confirms that the deletion of RT coding sequences from the viral DNA was effective in controlling the spread and replication of DNA in tissues. Another major advantage of the present vaccine is that booster injections of DNA could be administered indefinitely, irrespective of the nature of existing antiviral immunity.

Another feature that distinguishes the present vaccine from others being used is the fact that expression of viral genes in the present vaccine is regulated by the natural viral promoter. In this arrangement, the interaction between the viral DNA molecule and the transfected cell, in terms of persistence of viral DNA and subsequent expression of viral genes, simulates similar mechanisms of interaction between the replication-competent parental agents and the transfected cell. The one difference being that the new vaccines are not able to produce infectious particles and cause spreading infections.

It is contemplated that any suitable vector for delivering the DNA vaccine is within the scope of the present invention. It is also contemplated that deletion or disruption of genes in accordance with the teachings of the present invention can be accomplished by any of a variety of means well known in the art. Likewise, the various constructs described herein may be under the control of a wide variety of promoters. The examples described above include the use of the natural viral promoters as well as the CMV promoter. The use of specific promoters in the examples above should not be interpreted as limiting, however. Those skilled in the art may identify various promoters that would also be effective.

The parent of the present application, U.S. patent application Ser. No. 08/850,492, was directed to the invention of certain live virus HIV vaccines. The disclosure of the present case is incorporated herein by reference.

Various embodiments of the present invention have been described above. The examples provided in this disclosure are not intended to in any way limit the scope of the invention. Various additional modifications to the present invention will be readily apparent to those skilled in the art once such persons skilled in the art have obtained the information disclosed herein.

1. A vaccine for immunization against HIV comprising an isolated DNA molecule having a sequence encoding a plurality of viral proteins capable of stimulating an immune response against HIV, wherein the combination of viral proteins is rendered nonpathogenic by altering the DNA molecule such that it is unable to encode at least one functional protein selected from the group consisting of Nef, Vpu and reverse transcriptase.

2. A vaccine for immunization against HIV comprising an isolated DNA molecule having a sequence encoding a plurality of viral proteins capable of stimulating an immune response against HIV, wherein the combination of viral proteins is rendered nonpathogenic by altering the DNA molecule such that it is unable to encode a functional reverse transcriptase protein.

3. A vaccine for immunization against HIV comprising an isolated DNA molecule having a sequence encoding a plurality of viral proteins capable of stimulating an immune response against HIV, wherein the combination of viral proteins is rendered nonpathogenic by altering the DNA molecule such that it is unable to encode a functional Nef protein.

4. A vaccine for immunization against HIV comprising an isolated DNA molecule having a sequence encoding a plurality of viral proteins capable of stimulating an immune response against HIV, wherein the combination of viral proteins is rendered nonpathogenic by altering the DNA molecule such that it is unable to encode a functional Vpu protein.

5. The vaccine of claim 2 wherein said DNA molecule is altered by at least a partial deletion of a reverse transcriptase gene.

6. The vaccine of claim 2 wherein the altered DNA molecule has a nonfunctional reverse transcriptase gene having the same number of nucleotides as a functional, unaltered reverse transcriptase gene.

7. The vaccine of claim 2 wherein said DNA further comprises a 3′ LTR sequence and said DNA molecule has been further altered by at least a partial deletion of the 3′ LTR sequence.

8. The vaccine of claim 3 wherein said DNA molecule is altered by at least a partial deletion of a nef gene.

9. The vaccine of claim 3 wherein the altered DNA molecule has a nonfunctional nef gene having the same number of nucleotides as a functional, unaltered nef gene.

10. The vaccine of claim 3 wherein said DNA further comprises a 3′ LTR sequence and said DNA molecule has been further altered by at least a partial deletion of the 3′ LTR sequence.

11. The vaccine of claim 4 wherein said DNA molecule is altered by at least a partial deletion of a vpu gene.

12. The vaccine of claim 4 wherein said DNA further comprises a 3′ LTR sequence and said DNA molecule has been further altered by at least a partial deletion of the 3′ LTR sequence.

13. The vaccine of claim 1 wherein said DNA further comprises a 3′ LTR sequence and said DNA molecule has been further altered by at least a partial deletion of the 3′ LTR sequence.

14. The vaccine of claim 1, wherein the DNA molecule is derived from an HIV virus having a protein capable of downregulating CD4 levels in vivo, and the combination of viral proteins is rendered nonpathogenic by disrupting the ability of said DNA molecule to encode for the protein capable of downregulating CD4 levels in vivo.

15. The vaccine of claim 1, wherein the DNA molecule is derived from an HIV virus having a protein essential to the ability of the HIV virus to induce disease, and the combination of viral proteins is rendered nonpathogenic by disrupting the ability of the DNA molecule to encode for the protein essential to the ability of the HIV virus to induce disease.

16. The vaccine of any one of claims 1 through 15 further comprising a pharmaceutically acceptable carrier.

17. The vaccine of any one of claims 1 through 15 further comprising a natural HIV promoter sequence.

18. The vaccine of any one of claims 1 through 15 further comprising a CMV promoter sequence.

19. A DNA immunogenic composition derived from a viral genome coding for at least one protein capable of providing an immune response against HIV and having a 5′ long-terminal repeat and a 3′ long-terminal repeat, wherein the ability of the DNA immunogenic composition to integrate into a host genome has been destroyed by disruption of the 3′ long-terminal repeat.

20. A DNA immunogenic composition comprising the nucleotide sequence of SEQ ID NO:1.

21. A DNA immunogenic composition comprising the nucleotide sequence of SEQ ID NO:2.

22. A DNA immunogenic composition comprising the nucleotide sequence of SEQ ID NO:3.

23. A DNA immunogenic composition comprising the nucleotide sequence of SEQ ID NO:5.

24. A DNA immunogenic composition comprising the nucleotide sequence of SEQ ID NO:6.

25. The DNA immunogenic composition of any one of claims 20 through 24 further comprising a suitable vector.

26. The DNA immunogenic composition of any one of claims 20 through 24 further comprising a vector having the nucleotide sequence of SED ID NO:4.

27. A DNA immunogenic composition comprising a nucleotide sequence comprising:
(a) the 5′ LTR of SIV;
(b) the gag gene of SIV;
(c) the pro gene of SIV;
(d) the int gene of SIV;
(e) the vif gene of SIV;
(f) the vpr gene of SIV;
(g) the vpx gene of SIV;
(h) the rt gene of SIV wherein the rt gene of SIV has been disrupted;
(i) the env gene of HIV;
(j) the vpu gene of HIV;
(k) the nef gene of SIV; and
(l) the 3′ long-terminal repeat of SIV.

28. A DNA immunogenic composition according to claim 27 wherein said vpu gene has been disrupted.

29. A DNA immunogenic composition according to claim 27 wherein said nef gene has been disrupted.

30. A DNA immunogenic composition according to claim 27 wherein said 3′ long terminal repeat has been disrupted.

31. A DNA immunogenic composition according to claim 27 wherein said vpu and nef genes have been disrupted.

32. A DNA immunogenic composition according to claim 27 wherein said vpu and nef genes have been disrupted and further wherein said 3′0 long terminal repeat has been disrupted.

33. A DNA immunogenic composition comprising a nucleotide sequence comprising:
(a) the 5′ LTR of SIV;
(b) the gag gene of SIV;
(c) the pro gene of SIV;
(d) the int gene of SIV;
(e) the vif gene of SIV;
(f) the vpr gene of SIV;
(g) the vpx gene of SIV;
(h) the rt gene of SIV wherein the rt gene of SIV has been disrupted;
(i) the env gene of HIV;
(j) the vpu gene of HIV;
(k) the nef gene of SIV; and
(l) an SV 40 polyadenylation sequence.

34. A DNA immunogenic composition according to claim 33 wherein said vpu gene has been disrupted.

35. A DNA immunogenic composition according to claim 33 wherein said nef gene has been disrupted.

36. A DNA immunogenic composition according to claim 33 wherein said vpu and nef genes have been disrupted.

37. A DNA immunogenic composition comprising a nucleotide sequence comprising:
(a) the 5′0 LTR of SIV;
(b) the gag gene of HIV;
(c) the pro gene of HIV;
(d) the int gene of SIV;
(e) the vif gene of SIV;
(f) the vpr gene of SIV;
(g) the vpx gene of SIV;
(h) the rt gene of SIV wherein the rt gene of SIV has been disrupted;
(i) the env gene of HIV;
(j) the vpu gene of HIV;
(k) the nef gene of SIV; and
(l) an SV 40 polyadenylation sequence.

38. A DNA immunogenic composition according to claim 37 wherein said vpu gene has been disrupted.

39. A DNA immunogenic composition according to claim 37 wherein said nef gene has been disrupted.

40. A DNA immunogenic composition according to claim 37 wherein said vpu and nef genes have been disrupted.

41. A method of providing vaccination against HIV comprising administering to a recipient the DNA composition of any one claims 1 through 15, 19 through 24, or 27 through 40.

42. A recombinant virus wherein the DNA of said recombinant virus comprises SIV LTR, gag, pol and nef genes and HIV-1 env, tat, and rev genes, and a nonfunctional vpu gene from HIV-1, wherein the vpu gene is rendered nonfunctional by at least a partial deletion of the vpu gene and further wherein said nonfunctional vpu gene does not have the same number of nucleotides as a functional HIV-1 vpu gene.

43. The recombinant virus of claim 42 wherein said nef gene is rendered nonfunctional by at least a partial deletion of said nef gene.

44. A DNA construct comprising SIV LTR, gag, pol and nef genes and HIV-1 env, tat, and rev genes, and a nonfunctional vpu gene from HIV-1, wherein the vpu gene is rendered nonfunctional by at least a partial deletion of the vpu gene and further wherein said nonfunctional vpu gene does not have the same number of nucleotides as a functional HIV-1 vpu gene.

45. The DNA construct of claim 44 wherein said nef gene is rendered nonfunctional by at least a partial deletion of said nef gene.

46. An HIV-1/HIV-2 Chimeric virus wherein the DNA of the Chimeric virus comprises HIV-2 LTR, gag, pol, and nef genes and HIV-1 env, tat and rev genes and, optionally, an HIV-1 vpu gene, wherein said vpu gene if present is rendered nonfunctional.

47. The Chimeric virus of claim 46, wherein the vpu gene if present has been rendered nonfunctional by at least a partial deletion of said vpu gene.

48. A method for the creation of an effective vaccine for conveying immunity to HIV-1 virus comprising manipulating the HIV-1 virus to impede its ability to effectively replicate and/or otherwise accumulate in the infected/inoculated host.

49. A method as in claim 48 wherein the manipulation is the interference with the activity of the vpu gene or gene product of the virus.

50. A method for the treatment of currently infected HIV-1 positive patients comprising administering agents that will interfere with the HIV-1 vpu gene or gene products wherein such agents can be chemical, antibody-based, or other form of bioavtive molecule.

51. A method for the treatment of currently infected HIV-1 positive patients comprising administering agents that will interfere with the HIV-1 reverse transcriptase or gene products wherein such agents can be chemical, antibody-based, or other form of bioavtive molecule.
